(19) United States
(12) Patent Application Publication
(10) Pub. No.: US 2017/0204045 A1
(43) Pub. Date: Jul. 20, 2017

(54) NOVEL LACTIC ACID RECOVERY PROCESS

(71) Applicant: ARCHER DANIELS MIDLAND COMPANY, Decatur, IL (US)

(72) Inventor: Jose Leboreiro, Champaign (MX)

(73) Assignee: Archer Daniels Midland Company, Decatur, IL (US)

(21) Appl. No.: 15/324,898

(22) PCT Filed: Jul. 10, 2015
(86) PCT No.: PCT/US15/39957

§ 371 Date: Jan. 9, 2017

Related U.S. Application Data
(60) Provisional application No. 62/022,784, filed on Jul. 10, 2014.

(51) Int. Cl. C07C 67/54; C07C 67/08; C12P 7/56; C07C 67/03

(52) U.S. Cl. CPC C07C 67/54; C07C 67/03; C07C 67/08; C12P 7/56

(57) ABSTRACT

A novel two step distillation process for recovering lactic acid and ethyl lactate from still bottoms is described. In comparison to conventional distillation processes, this process involves a reaction and a second distillation which converts the lactic acid monomer and other lactic acid species that remain in the still bottoms after a first, conventional distillation step into ethyl lactate by simultaneously esterifying and transesterifying all lactic acid species present in the still bottoms.

### CROSS-REFERENCE TO RELATED APPLICATION(S)

This application claims the benefit of U.S. Provisional Patent Application No. 62/022,784, filed Jul. 10, 2014, the contents of which are incorporated by this reference.

### FIELD OF THE INVENTION

The present invention relates to the purification of lactic acid and ethyl lactate from other lactic acid species obtained from processing a fermentation broth obtained by using a microorganism to produce lactic acid. In particular, the present invention describes a novel two step distillation process for recovering lactic acid and ethyl lactate from still bottoms containing other lactic acid species that were in the past considered waste products.

### BACKGROUND OF THE INVENTION

Lactic acid (2-hydroxypropionic acid) is an organic acid that can be produced synthetically, or naturally, by living organisms. Commercially, natural production by fermentation using microorganisms is a preferred method, especially when there is an abundance of carbohydrates to use as a carbon source. Lactic acid has become a valuable commodity over the last several decades, with applications in the food, pharmaceutical and cosmetic industries. Recently, the use of lactic acid has broadened into industrial applications, such as being used in the production of biodegradable and renewable raw material based polymers. And as applications for lactic acid use has broadened, so has its demand and the need to optimize every step of its production. Intense research has been done for years on these optimizations, from optimizing the production organisms using genetically engineering techniques to optimization of the physical processes used for purification of the fermentation products.

Fermentation of lactic acid is a widely varied discipline and can be done in many ways with many different organisms. Some examples of such organisms that are known in the art include, but is not limited to varying species of the genera Lactobacillus, Pediococcus, Lactococcus, Streptococcus, Saccharomyces, Schizosaccharomyces, and Rhizopus. Once the fermentation process is complete, the final broth is put through a series of purification steps that may or may not include, the filtration of cell mass, evaporation of water, acid precipitation, carbon filtering, evaporation, distillation, and ion exchange treatment. Examples of these lactic acid purification processes and steps can be found in U.S. Pat. No. 2,350,370; U.S. Pat. No. 6,489,508; U.S. Pat. No. 5,681,728; U.S. Pat. No. 7,244,596.

There are two common ways to purify lactic acid from a fermentation broth. For the purposes of this disclosure, one will be referred to as a molecular distillation and the other will be referred to as a reactive distillation. In molecular distillation, the fermentation broth is evaporated to a low water concentration and further distilled by a wiped film evaporator and a short path distillation column. The majority of the lactic acid is distilled in the overheads of the short path distillation. The other lactic acid species leave the distillation column through the bottoms. The bottoms also contains monomeric lactic acids, lactic oligomers, ethyl lactate, water, glycerol, succinic acid, fumaric acid, mail acid and esters of thereof and other minor impurities including ionic species as well as high boiling compounds. All lactic species (in any chemical form) in the bottoms contribute to yield loss in the production process. The term ‘other lactic acid species’ herein refers to any combination of the following: lactic acid, lactate, lactide, ethyl lactate, esters of glycerol, lactates of inorganic salts, and lactic acid oligomers in acid or ethyl ester form.

The other common purification process to separate lactic acid is by a reactive distillation whereby lactic acid is esterified with an alcohol using an acid catalyst simultaneously during distillation. Again, the esterified lactic acid boils with the overhead and the high boiling impurities leave the column through the bottoms. The main alcohols used for this purpose are methanol and ethanol. Sulfuric acid is added into the distillation apparatus as a catalyst for the reaction between lactic acid and the alcohol. Lactic acid esters, such as ethyl lactate, can be readily converted back to free lactic acid and the alcohol by simple acid or base hydrolysis. The majority of lactic acid is converted to ethyl lactate; however, some lactic acid is esterified with glycerol and another portion forms oligomers of lactic acid which can be in the acid or alcohol esterified form. These items end up in the bottoms as described herein before, which is a waste stream of the distillation process.

The present invention focuses on improvements to the distillation process for recovery of lactic acid and ethyl lactate from other lactic acid species produced incident to the purification process to increase the total percentage yield of lactic acid or useful esters thereof.

### SUMMARY OF THE INVENTION

Described herein is a method of recovering ethyl lactate from a lactic acid fermentation process, comprising, performing a first distillation of a crude lactic acid containing fermentation broth and obtaining a first purified fraction containing lactic acid and leaving a still bottoms fraction containing other lactic acid species; adding sulfuric acid and ethanol to the still bottoms fraction to transesterify the other lactic acid species in the still bottoms to ethyl lactate forming a reacted still bottoms fraction; and performing a second distillation that distills the reacted still bottoms fraction to obtain a second purified fraction containing ethyl lactate. In some embodiments, the fermentation process can use a Schizosaccharomyces sp. microorganism to produce lactic acid. In a particular embodiment, the microorganism can be a Schizosaccharomyces pombe. Other lactic acid species, as defined herein are selected from the group consisting of lactic acid, lactate, lactide, ethyl lactate, esters of glycerol, lactates of inorganic salts, and lactic acid oligomers in acid or ethyl ester form. This method can recover up to 95% of the other lactic acid species in said first distillation still bottoms as ethyl lactate. Up to 2 volumes of water can be added to the still bottoms of the first distillation process, but the addition of water is not necessary and may reduce recovery. Typically, water will be present when the added ethanol is not anhydrous. Ethanol containing 7.4% water was used in some exemplary embodiments and the yield of ethyl lactate from other lactic acid species in the first still bottoms was up to 95%, which was comparable to when anhydrous ethanol was used. In an exemplary embodiment, 0.5 volumes of water and 2 volumes of ethanol per volume of still bottoms was added and the yield of ethyl lactate from the still bottoms was 31%. The reactions can be performed with 0.01 to 4 volumes of ethanol and 0.001 to 0.06 volumes of sulfuric acid added to 1 part still bottoms from the first distillation. In more typical embodiments, 0.05 to 2 volumes of ethanol and 0.005 to 0.04 volumes of sulfuric acid are added to 1 part still bottoms from the first distillation. The transesterification during the second distillation can be performed at 60° C.-120° C. In exemplified embodiments the reaction was performed at 90° C. The reaction time will vary according to volume, in exemplified embodiments the reactive distillations were performed for as little as 15 mins to as long as 24 hours with a total distillation volume inclusive of still bottoms, ethanol, water and sulfuric acid of about 10 ml to about 2 L.

Additionally described herein is a method of recovering ethyl lactate from a lactic acid fermentation process, comprising, performing a first distillation of a crude lactic acid containing fermentation broth in the presence of ethanol and sulfuric acid and obtaining a first purified fraction containing ethyl lactate and leaving a still bottoms fraction containing other lactic acid species; adding ethanol to the still bottoms fraction to transesterify the other lactic acid species in the still bottoms to ethyl lactate forming a reacted still bottoms fraction; and performing a second distillation that distills the reacted still bottoms fraction to obtain a second purified fraction of ethyl lactate. In some embodiments of this method, the fermentation process can use a microorganism selected from the group consisting of Rhizopus sp. and Schizosaccharomyces sp. to produce lactic acid. In some specific embodiments, the microorganism can be Rhizopus oryzae or Schizosaccharomyces pombe. Additional sulfuric acid can be added to the bottoms of the first distillation before the second distillation, but is not necessary. Again, the other lactic acid species are selected from the group consisting of lactic acid, lactate, lactide, ethyl lactate, esters of glycerol, lactates of inorganic salts, and lactic acid oligomers in acid or ethyl ester form. Temperatures, times, and relative volumes of reactants can be the same as described above.

In the broadest embodiments of any of the forgoing inventions, at least 30% of the other lactic acid species in the still bottoms is recovered as ethyl lactate. In more desirable embodiments at least 50% of the other lactic acid species in the still bottoms is recovered as ethyl lactate. In still more desirable embodiments at least 75% of the other lactic acid species in the still bottoms is recovered as ethyl lactate. In the most desirable embodiments 85% to 95% of the other lactic acid species in the still bottoms is recovered as ethyl lactate. In some exemplary embodiments 92- 95% of the other lactic acid species in the still bottoms is recovered as ethyl lactate.

### BRIEF DESCRIPTION OF THE DRAWINGS

FIG. 1: A graph showing the progression of the reaction as described in the Example 1.

FIG. 2: A graph showing the progression of the reaction as described in the Example 4.

FIG. 3: A flow diagram of a Reactive Distillation

FIG. 4: A flow diagram of a Molecular Distillation

### DETAILED DESCRIPTION OF THE INVENTION

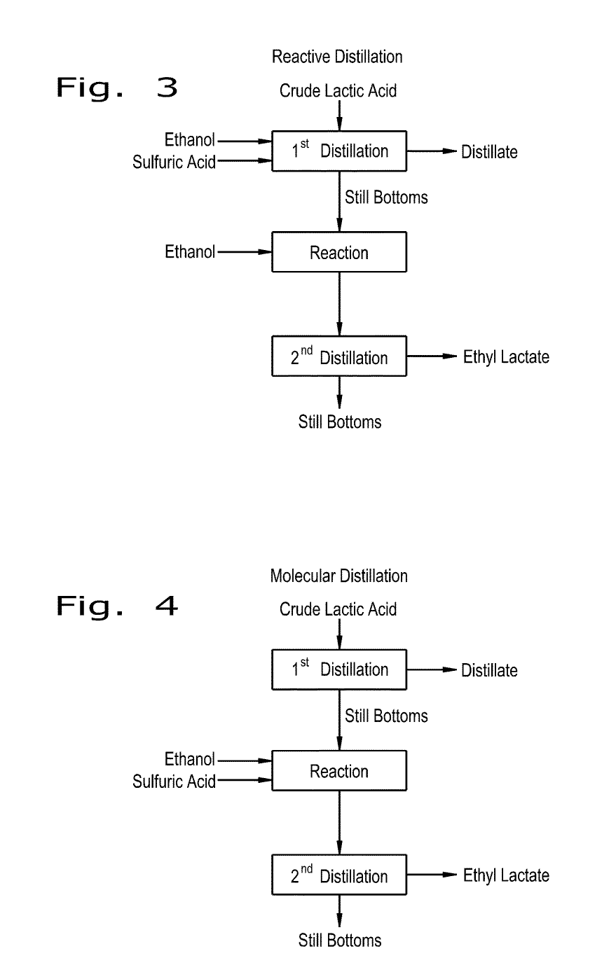

The most advantageous contribution of the lactic acid distillation methods described herein is the ability to recover more lactic acid (or ethyl lactate) from the still bottoms remaining after conventional distillation of a lactic acid fermentation broth. This new process involves a reaction and a second distillation which converts the lactic acid monomer and other lactic acid species that remain in the bottoms after a first distillation step to ethyl lactate by simultaneously esterifying and transesterifying all other lactic acid species with ethanol. The ethanol added to this reaction step may be process ethanol or anhydrous ethanol. Process ethanol is hereby defined as ethanol that has not been purified. Process ethanol is ethanol that may include other components such as, but not limited to, water, glycerol, diethyl ether, lactic acid, formic acid, acetic acid, succinic acid, maleic acid, fumaric acid, and ethyl ester of these organic acids. Anhydrous ethanol is hereby defined as purified ethanol that is 200 proof ethanol or ≧99.5%. The use of anhydrous ethanol leads to an improvement in the transesterification conversion. Sulfuric acid can be used to catalyze the esterification and transesterification reaction of lactic acid monomer and other lactic acid species. If the first distillation is a reactive distillation, which involves sulfuric acid, the sulfuric acid leaves the reacted distillation column through the bottoms, in which case no additional catalyst has to be added in the second distillation. This is because sulfuric acid is added in the initial steps of a reactive distillation as seen in FIG. 3 and inevitably, residual sulfuric acid will remain in the bottoms. However, supplementing the second distillation with additional catalyst may increase the reaction rate of the second distillation.

The esterification and transesterification reaction can be carried out in a variety of reactor designs including batch, continuous stirred tank reactor (CSTR), and tubular reactor. The tubular reactor may operate in the laminar, transitional, or turbulent flow regime.

In the case of the molecular distillation as seen in FIG. 4, sulfuric acid has to be added to catalyze the esterification and transesterification of lactic acid monomer and other lactic acid species during the second distillation because no sulfuric acid is added in the initial steps of a molecular distillation process. The reacted bottoms can be distilled to recover the ethyl lactate and unreacted ethanol. The use of this invention will allow the recovery of lactic acid and other lactic acid species lost in the bottoms of the distillation column.

### EXAMPLES

The present invention is further demonstrated by the non-limiting examples that follow. In each of the examples, a lactic acid fermentation broth was obtained, and subjected to either a reactive distillation with ethanol or a molecular distillation to obtain a first distillation bottoms

### Example 1

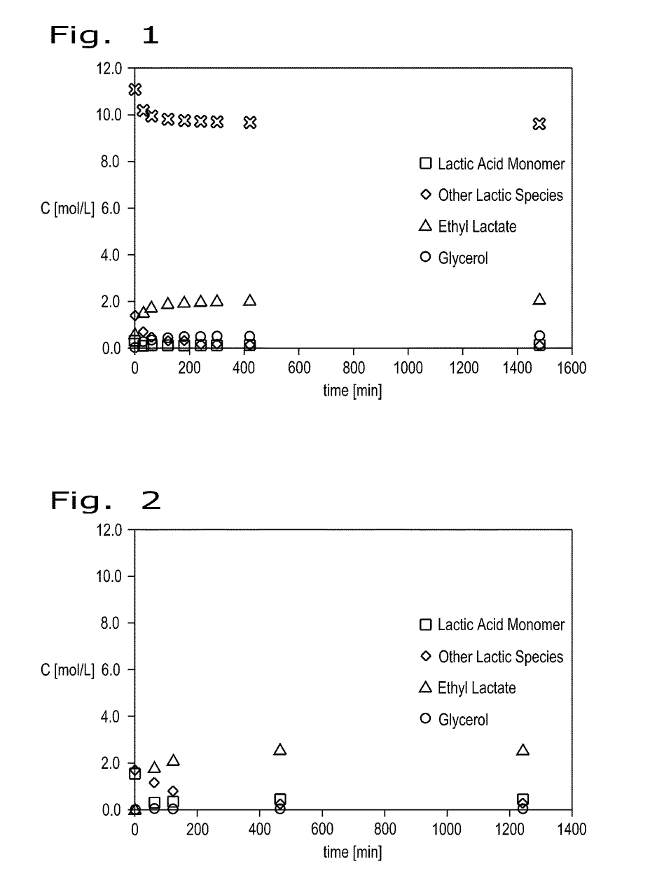

A mixture of 1 part distillation bottoms from a reactive distillation and 1 part anhydrous ethanol were blended in a batch reactor. The reactor was heated to 90° C. for 24 h. Samples from the reactor were taken periodically and analyzed for lactic acid monomer, other lactic acid species, ethyl lactate, glycerol, and ethanol. The composition of the 1 part bottoms and 1 part ethanol mixture in mol/L before the reaction and after the reaction can be found in Table 1. The ethyl lactate and glycerol concentration increases while the concentration of other lactic acid species decreases. The increase in the concentration of glycerol is due to the transesterification of the lactic esters of glycerol. A conversion of only the other lactic acid species to ethyl lactate equal to 0.92 mole fraction was obtained. A total lactic conversion, which for the purposes of this description will be defined as a conversion of the lactic acid monomer and the other lactic acid species (combined) to ethyl lactate equal to 0.86 mole fraction was obtained. The progression of this reaction can be seen in FIG. 1.

TABLE 1

| Component of Mixture | Before Reaction mol/L | After Reaction mol/L |
|---|---|---|
| Lactic Acid Monomer | 0.2997 | 0.1320 |
| Other Lactic Acid | 1.4002 | 0.1074 |
| species | | |
| Ethyl Lactate | 0.6316 | 2.0961 |
| Glycerol | 0.0276 | 0.5149 |
| Ethanol | 11.1029 | 9.6280 |

### Example 2

A mixture of 1 part distillation bottoms from a reactive distillation and 1 part of ethanol with 7.4% wt. water were blended in a batch reactor. The reactor was heated to 120° C. for 24 h. Samples from the reactor were taken periodically and analyzed for lactic acid monomer, other lactic acid species, ethyl lactate, glycerol, and ethanol. The composition of the 1 part bottoms and 1 part ethanol/water mixture in mol/L before the reaction and after the reaction can be found in Table 2. A conversion of other lactic acid species to ethyl lactate equal to 0.95 mole fraction and a total lactic conversion (including lactic acid monomer) to ethyl lactate equal to 0.75 mole fraction was obtained.

TABLE 2

| Component of Mixture | Before Reaction mol/L | After Reaction mol/L |
|---|---|---|
| Lactic Acid Monomer | 0.2997 | 0.3412 |
| Other Lactic Acid | 1.3619 | 0.0688 |
| species | | |
| Ethyl Lactate | 0.6700 | 1.9114 |
| Glycerol | 0.0276 | 0.5029 |
| Ethanol | 10.0296 | 8.8122 |

### Example 3

A mixture of 1 part distillation bottoms from a reactive distillation and 1 part of ethanol with 7.4% wt. water were blended and fed to a continuous stir tank reactor. The reactor was heated to 90° C. and operated with a 1 h residence time. Samples from the reactor were taken periodically and analyzed for lactic acid monomer, other lactic acid species, ethyl lactate, glycerol, and ethanol. The composition of the 1 part bottoms and 1 part ethanol/water mixture in mol/L before the reaction and after the reaction can be found in Table 3. A conversion of other lactic acid species to ethyl lactate equal to 0.62 mole fraction and a total lactic conversion (including lactic acid monomer) to ethyl lactate equal to 0.54 mole fraction was obtained.

TABLE 3

| Component of Mixture | Before Reaction mol/L | After Reaction mol/L |
|---|---|---|
| Lactic Acid Monomer | 0.3096 | 0.2615 |
| Other Lactic Acid | 1.4464 | 0.5432 |
| species | | |
| Ethyl Lactate | 0.6905 | 1.6418 |
| Glycerol | 0.0773 | 0.3355 |
| Ethanol | 9.9608 | 9.0096 |

### Example 4

A mixture of 1 part esterification bottom and 1 part ethanol were blended and fed to a tubular reactor at 90° C. and given a residence time of 60 minutes, conversion of other lactic acid species to ethyl lactate equal to 0.64 mole fraction and a total lactic conversion (including lactic acid monomer) to ethyl lactate equal to 0.67 mole fraction was obtained. The composition of the 1 part bottoms and 1 part ethanol in mol/L before the reaction and after the reaction can be found in Table 4.

TABLE 4

| Component of Mixture | Before Reaction mol/L | After Reaction mol/L |
|---|---|---|
| Lactic Acid Monomer | 0.2997 | 0.0673 |
| Other Lactic Acid | 1.3619 | 0.4889 |
| species | | |
| Ethyl Lactate | 0.6700 | 1.7754 |
| Glycerol | 0.0276 | 0.3207 |
| Ethanol | 10.0296 | 8.9242 |

### Example 5

A mixture of 1 part wiped film evaporator bottoms from a molecular distillation, 1 part of anhydrous ethanol, and 0.04 parts of sulfuric acid were blended and fed to a batch reactor. The reactor was heated to 90° C. for 1245 min. Samples from the reactor were taken periodically and analyzed for lactic acid monomer, other lactic acid species, ethyl lactate, glycerol, and ethanol. The composition of the bottoms/ethanol/sulfuric acid mixture in mol/L before the reaction and after the reaction can be found in Table 5. The ethyl lactate and glycerol concentration increases while the concentration of other lactic acid species decreases. The increase in the concentration of glycerol is due to the transesterification of the lactic esters of glycerol. A conversion of other lactic acid species to ethyl lactate equal to 0.86 mole fraction and a total lactic conversion (including lactic acid monomer) to ethyl lactate equal to 0.80 mole fraction was obtained. The progression of this reaction can be seen in FIG. 2.

TABLE 5

| Component of Mixture | Before Reaction mol/L | After Reaction mol/L |
|---|---|---|
| Lactic Acid Monomer | 2.4145 | 0.3957 |
| Other Lactic Acid | 1.6737 | 0.2286 |
| species | | |
| Ethyl Lactate | 0.0000 | 2.5514 |
| Glycerol | 0.0008 | 0.0167 |
| Ethanol | 13.4241 | 10.9265 |

### Example 6

A mixture of 1 part short path distillation bottoms from a molecular distillation, 0.5 parts of water, 2 parts of anhydrous ethanol, and 0.06 parts of sulfuric acid were blended and fed to a batch reactor. The reactor was heated to 90° C. for 1245 min. The composition of the bottoms/water/ethanol/sulfuric acid mixture in mol/L before the reaction and after the reaction can be found in Table 6. A conversion of other lactic acid species to ethyl lactate equal to 0.31 mole fraction and a total lactic conversion (including lactic acid monomer) to ethyl lactate equal to 0.34 mole fraction was obtained.

TABLE 6

| Component of Mixture | Before Reaction mol/L | After Reaction mol/L |
|---|---|---|
| Lactic Acid Monomer | 0.5146 | 0.2663 |
| Other Lactic Acid | 1.6058 | 1.0959 |
| species | | |
| Ethyl Lactate | 0.0000 | 0.7459 |
| Glycerol | 0.0220 | 0.0477 |
| Ethanol | 13.0981 | 12.3771 |

What is claimed is:

1. A method of recovering ethyl lactate from a lactic acid fermentation process, comprising,
a. performing a first distillation of a crude lactic acid containing fermentation broth and obtaining a first purified fraction containing lactic acid and leaving a still bottoms fraction containing other lactic acid species;
b. adding sulfuric acid and ethanol to the still bottoms fraction to transesterify the other lactic acid species in the still bottoms to ethyl lactate forming a reacted still bottoms fraction; and
c. performing a second distillation that distills the reacted still bottoms fraction to obtain a second purified fraction containing ethyl lactate.

2. The method of claim 1, wherein said fermentation process uses a Schizosaccharomyces sp. microorganism to produce lactic acid.

3. The method of claim 2, wherein said microorganism is Schizosaccharomyces pombe.

4. The method of claim 1 wherein said other lactic acid species is selected from the group consisting of lactic acid, lactate, lactide, ethyl lactate, esters of glycerol, lactates of inorganic salts, and lactic acid oligomers in acid or ethyl ester form.

5. The method of claim 1 wherein 85% to 95% of the other lactic acid species in said first distillation still bottoms is recovered as ethyl lactate.

6. The method of claim 1 wherein water is added to the still bottoms of the first distillation process.

7-8. (canceled)

9. The method of claim 1 where 0.05 to 2 volumes of ethanol and 0.005 to 0.04 volumes of sulfuric acid are added to 1 part still bottoms from the first distillation.

10. The method of claim 1 where 0.01 to 4 volumes of ethanol and 0.001 to 0.06 volumes of sulfuric acid and up to 2 volumes of water are added to 1 part still bottoms from the first distillation.

11. The method of claim 1 wherein said transesterification is performed at 60° C.-120° C.

12-13. (canceled)

14. A method of recovering ethyl lactate from a lactic acid fermentation process, comprising,
a. performing a first distillation of a crude lactic acid containing fermentation broth in the presence of ethanol and sulfuric acid and obtaining a first purified fraction containing ethyl lactate and leaving a still bottoms fraction containing other lactic acid species;
b. adding ethanol to the still bottoms fraction to transesterify the other lactic acid species in the still bottoms to ethyl lactate forming a reacted still bottoms fraction; and
c. performing a second distillation that distills the reacted still bottoms fraction to obtain a second purified fraction of ethyl lactate.

15. The method of claim 14, wherein further sulfuric acid is added to step b before said second distillation.

16. The method of claim 14, wherein said fermentation process uses a microorganism selected from the group consisting of Rhizopus sp. and Schizosaccharomyces sp. to produce lactic acid.

17. The method of claim 16, wherein said microorganism is Rhizopus oryzae.

18. The method of claim 16, wherein said microorganism is Schizosaccharomyces pombe.

19. The method of claim 14 wherein said other lactic acid species are selected from the group consisting of lactic acid, lactate, lactide, ethyl lactate, esters of glycerol, lactates of inorganic salts and lactic acid oligomers in acid or ethyl ester form.

20. The method of claim 14 wherein 85-95% of the other lactic acid species in said first distillation still bottoms is recovered as ethyl lactate.

21. The method of claim 14 wherein said transesterification is performed at 60° C.-120° C.

22. (canceled)

23. The method of claim 14 wherein the ethanol is selected from the group consisting of process ethanol, anhydrous ethanol and a mixture of process and anhydrous ethanol.

24. The method of claim 14 where 0.01 to 4 volumes of ethanol and 0.001 to 0.06 volumes of sulfuric acid are added to 1 part still bottoms of the first distillation process.

25. The method of claim 14 wherein said transesterification is carried out in a reactor selected from the group consisting a batch reactor, a continuous stirred tank reactor (CSTR) and a tubular reactor.
